(19) United States
(12) Patent Application Publication
(10) Pub. No.: US 2003/0004125 A1
(43) Pub. Date: Jan. 2, 2003

(54) Inducible gene

(76) Inventors: David G. Hirst, Belfast (GB); Tracey Robson, Belfast (GB); Jenny Isobel Worthington, Larne (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/177,093

(22) Filed: Jun. 21, 2002

Related U.S. Application Data
(60) Provisional application No. 60/300,294, filed on Jun. 22, 2001.

(30) Foreign Application Priority Data
Nov. 23, 2001 (GB) 0128135.1

(51) Int. Cl. A61K 48/00; C12N 15/01

(52) U.S. Cl. 514/44; 435/447

(57) ABSTRACT

A method to regulate the expression of a sequence encoding a gene using ionising radiation, wherein said method comprises the steps of:

a. constructing a vector including the promoter of the X-ray inducible gene WAF1 and a sequence encoding an inducible gene the expression of which is regulated by the promoter of the X-ray inducible gene WAF1,

b. delivering said vector to the target cells, tissue or organ,

c. modulating the level of expression of the inducible gene(s) by providing ionising radiation suitable to cause the promoter of WAF1 to effect the expression of the sequence encoding the inducible gene.

The method can be used in the treatment of tumours.

The present invention relates to a method for inducing a gene following a radiation dose capable of activating the promoter for the X-ray inducible gene WAF1. In particular the invention relates to the induction a gene the expression of which is regulated by the promoter of the X-ray inducible gene of WAF1.

The success of non-surgical methods of cancer treatment is dependent on the supply of blood to the tumour. It has been reported that damage to a single vessel can cause the death of thousands of dependent tumour cells, and only a small number of endothelial cells need to be affected to disrupt a vessel. It would therefore be advantageous to locally alter the tumour blood supply without causing damage to normal vasculature.

Studies using the NOS inhibitor L-NAME on tumour growth rate in rat shows that tumours are very dependent on Nitric Oxide to maintain their blood vessels in a dilated state and thus supply nutrients for growth.

At normal physiological conditions Nitric Oxide (NO) is a potent biological mediator with diverse physiological and pathophysiological functions. It can act as a vasodilator, neurotransmitter, antimicrobial effector molecule and immunomodulator.

Nitric Oxide is produced by one of three distinct isoforms of the Nitric Oxide Synthase (NOS) gene. The inducible isoform of the enzyme (iNOS) produces large quantities of NO in a Calcium independent manner.

Elevated levels of NOS have been detected in a number of human and mammalian tumour tissues, although more than one cell type may be expressing the gene. Increased expression of NOS may facilitate the delivery of oxygen and nutrients through the dilated microvessels to the rapidly proliferating tumour cells.

At high concentrations Nitric Oxide is a potent chemical radiosensitizer, with a senstizer enhancement ratio of 2.3, at a concentration of 1% it is almost as effective as oxygen. Further, at high concentrations, NO is anti-angiogenic and cytotoxic.

The induction of inducible Nitric Oxide Synthase (iNOS) in the vasculature has been suggested to act to aid the delivery of chemiotherapeutic agents through vasodilated vessels or enhance oxygen delivery to reduce the number of radioresistant hypoxic cells.

Similar enhancement has also been reported with Nitric Oxide donor drugs.

However, delivery of adequate concentrations of NO through either the expression of iNOS or the administration of NO donor drugs to tumours at the time of irradiation remains a problem, because of the potent systemic effects of NO.

Thus it would be advantageous to develop a strategy which provides increased levels of Nitric Oxide to tumour cells at the time of the irradiation.

Due to the ease of accessibility and transfer of the transgene to a specific site or to the entire vasculature, the vasculature is a unique target organ for gene therapy.

However, the main problem with gene therapy protocols is their lack of target specificity.

The poor target specificity of gene therapy as currently practised means that it would be difficult to use this technique to deliver adequate concentrations of NO to tumours due to the potent systemic effects of NO or NO donor compounds.

The recognised problem of lack of target specificity in gene therapy protocols means that a number of strategies for gene therapy have been devised to localise therapy to the desired area. These include the use of tissue-specific receptors and tissue-specific enhancers to control expression at the transcription level. Alternatively, inducible promoters can be employed to transcriptionally modulate gene expression. A number of inducible systems have been developed; a tetracycline-controlled expression system, and a progesterone antagonist-regulated promoter.

Further an inducible system using radiation-responsive consensus sequences from the early growth response (EGR) gene promoter ligated upstream from the TNF-α gene has been utilised. This inducible system was used in studies that reported an increase in intratumour production of TNF-α when nude mice were injected with the Egr-TNF construct and exposed to radiation. More recently it has been reported that the EGR-1 promoter can be activated by the more clinically relevant dose of 2 Gy.

The object of the present invention is to provide an improved means of regulating inducible gene expression by using ionising radiation.

Accordingly the present invention provides a method to regulate the expression of a sequence encoding a gene using ionising radiation, wherein said method comprises the steps of:

1. constructing a vector including the promoter of the X-ray inducible gene WAF1 and a sequence encoding an inducible gene the expression of which is regulated by the promoter of the X-ray inducible gene WAF1,

2. delivering said vector to the target cells, tissue or organ,

3. modulating the level of expression of the inducible gene(s) by providing ionising radiation suitable to cause the promoter of WAF1 to effect the expression of the sequence encoding the inducible gene.

Preferably the construct is delivered into cells by transfection.

Preferably the expression of the sequence encoding the inducible gene is regulated by the promoter of the X-ray inducible gene WAF1 encodes the inducible isoform of the Nitric Oxide Synthase enzyme.

Alternatively the expression of the sequence encoding the inducible gene is regulated by the promoter of the X-ray inducible gene WAF1 encodes the antisense gene to the gene which encodes the inducible isoform of the Nitric Oxide Synthase enzyme.

Preferably the level of expression of the sequence which encodes the inducible gene the expression of which is regulated by the promoter of the X-ray inducible gene WAF1 is modulated by providing X-rays at a clinically relevant dose.

Preferably the level of expression of the sequence encoding the inducible gene is modulated by providing X-rays between the range 2 Gy-30 Gy.

More preferably the level of expression of the sequence encoding the inducible gene is modulated by providing X-rays between the range 2 Gy-8 Gy.

Preferably the method includes administration of a cofactor.

Preferably the cofactor is tetrahydrobiopterin or an equivalent thereof.

The invention further provides the use of a vector comprising the promoter region for the X-ray inducible gene WAF1 and a sequence which encodes the inducible isoform of Nitric Oxide Synthase enzyme the expression of which is regulated by the promoter of the X-ray inducible gene WAF1.

Alternatively the sequence which encodes for the inducible isoform of Nitric Oxide Synthase enzyme is an artificial sequence homologous to that of the naturally occurring gene.

Preferably the method comprises administering at least two injections into a tumour to increase anti-tumour efficacy.

The invention further provides a method of treatment of a tumour said method comprising the steps of,

1 introducing a vector including the promoter for the X-ray inducible gene WAF1, and a sequence encoding an inducible gene the expression of which is regulated by the promoter of the X-ray inducible gene WAF1 into the tumour,

2 irradiating the tumour such that the promoter for the X-ray inducible gene WAF1 induces sufficient levels of gene expression of the sequence for the inducible gene to provide physiological effect

The invention further provides a kit for use in the treatment of tumours wherein the kit comprises a vector including the promoter for the X-ray inducible gene WAF1 and a sequence encoding an inducible gene, the expression of which is regulated by the promoter of the X-ray inducible gene WAF1.

Preferably the kit also comprises liposomes containing said vector such that the vector can be transfected into cells, tissues or organs.

More preferably the kit also comprises cofactors which aid the production of Nitric Oxide by inducible Nitric Oxide Synthase.

Preferably a cofactor included in the kit is the compound tetrahydrobiopterin, BH₄.

The invention also provides use of the construct and/or cofactor in the preparation of a medicament for the treatment of tumours.

An embodiment of the present invention will now be described, by way of example only, with reference to the accompanying figures in which;

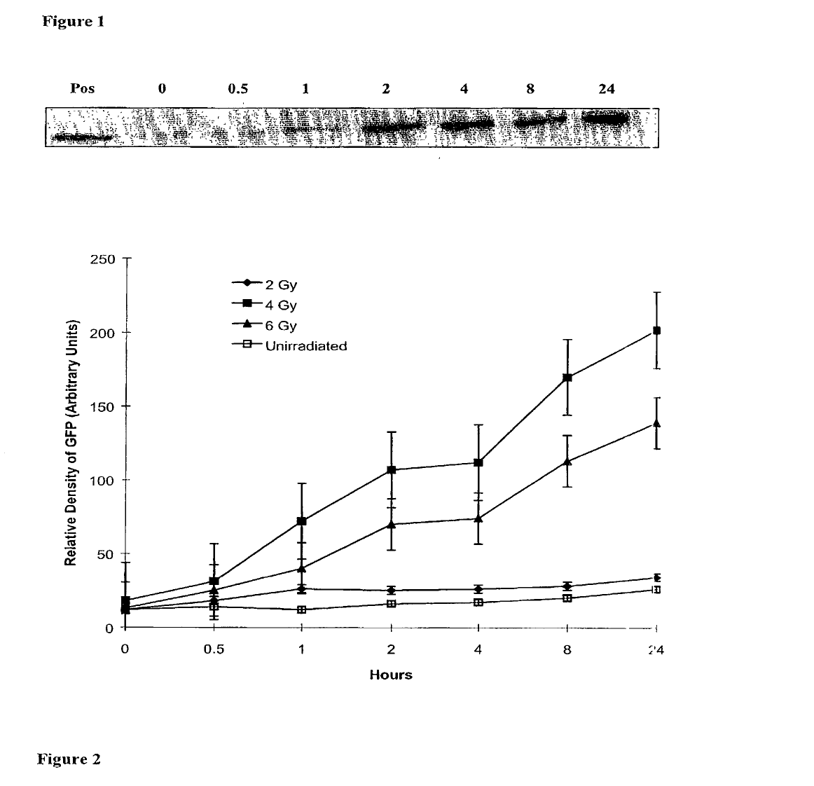

FIG. 1 shows a typical western blot analysis of GFP expression over time after exposure to 4 Gy,

FIG. 2 describes WAF1 promoter activity in HMEC-1 cells determined by Green Fluorescent Protein expression (GFP). Cells were harvested 0, 0.5, 1, 2, 4, 8, and 24 hours after irradiation. Each data point represents the mean of three experiments±s.e.,

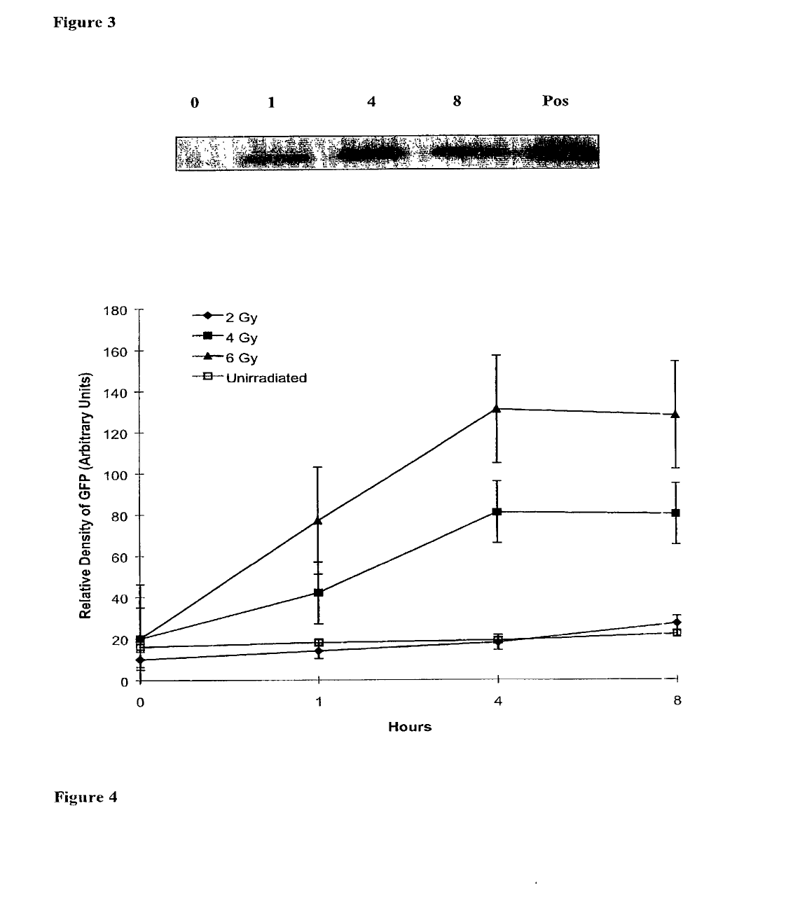

FIG. 3 shows a typical western blot analysis of GFP expression over time after exposure to 4 Gy,

FIG. 4 shows WAF1 promoter activity in rat tail artery segments determined by GFP (Green Fluroscent Protein) expression. Sections were homogenised 0, 1, 4 and 8 hours after irradiation. Each data point represents the mean of 3 experiments±s.e.,

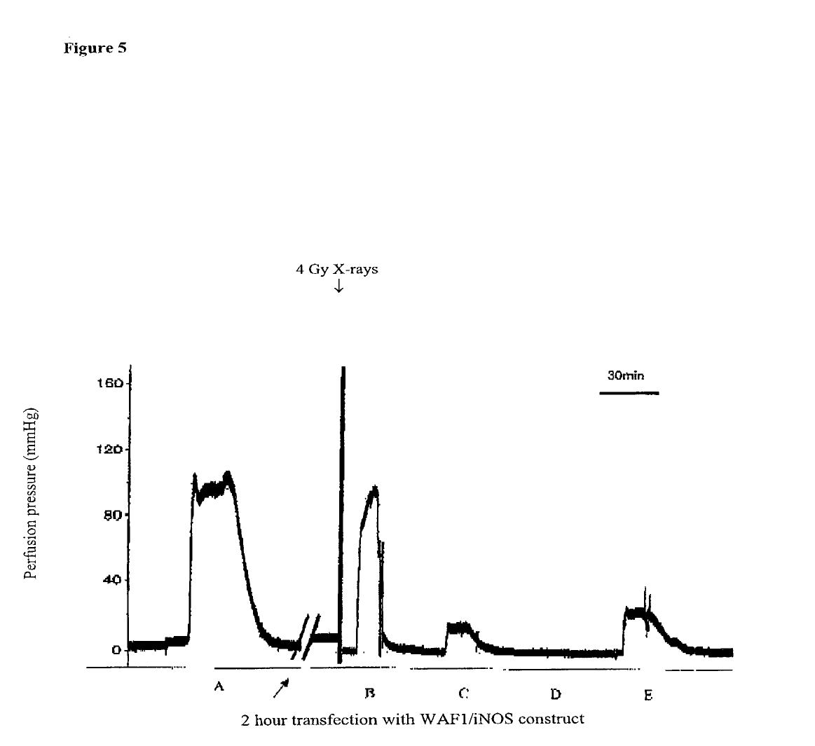

FIG. 5 shows a computer trace of phenylephrine PE (2×10⁻⁵ M) induced constriction of rat tail artery before and after transfection with WAF1/iNOS and exposure to 4 Gy X-rays. At each time point A, B, C, D and E, PE was perfused through each segment. Between points D and E the artery was perfused with the NOS inhibitor nitro-L-arginine for 1 hour,

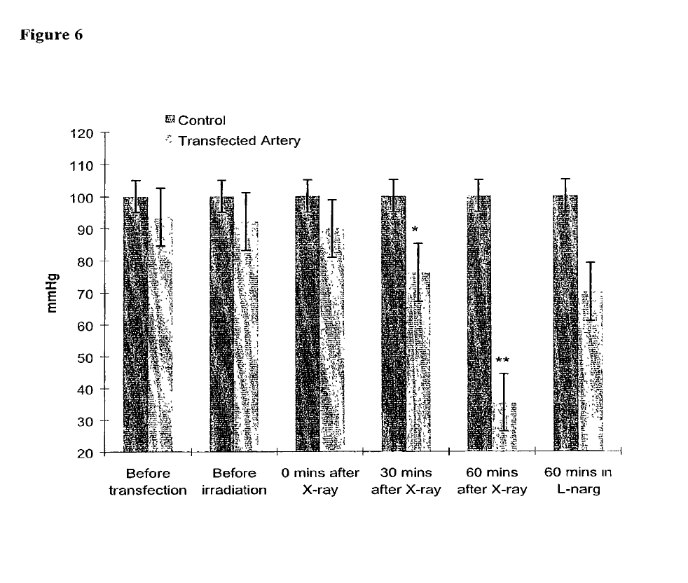

FIG. 6 shows PE (2×10⁻⁵M) induced constriction of rat tail artery segments before and after transfection with WAF1/iNOS and exposure to 4 Gy X-rays. The control artery was transfected with WAF1/iNOS construct but not irradiated. The data shown is the mean of 7 experiments ±s.e. * P<0.001 vs before transfection; ** P<0.001 vs before transfection,

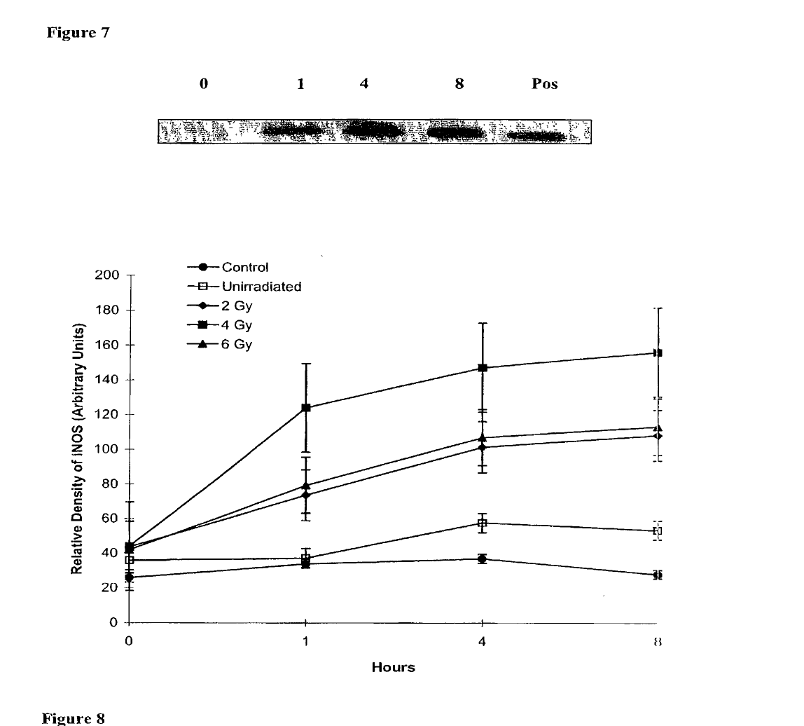

FIG. 7 shows a typical western blot analysis of iNOS expression over time,

FIG. 8 describes a time course of WAF1 promoter activity in rat tail artery segments demonstrated by iNOS protein expression. Sections were homogenised 0, 1, 2, 4, 8 hours after irradiation. Control segments were neither transfected or irradiated. Each data point represents the mean of three experiments±s.e.,

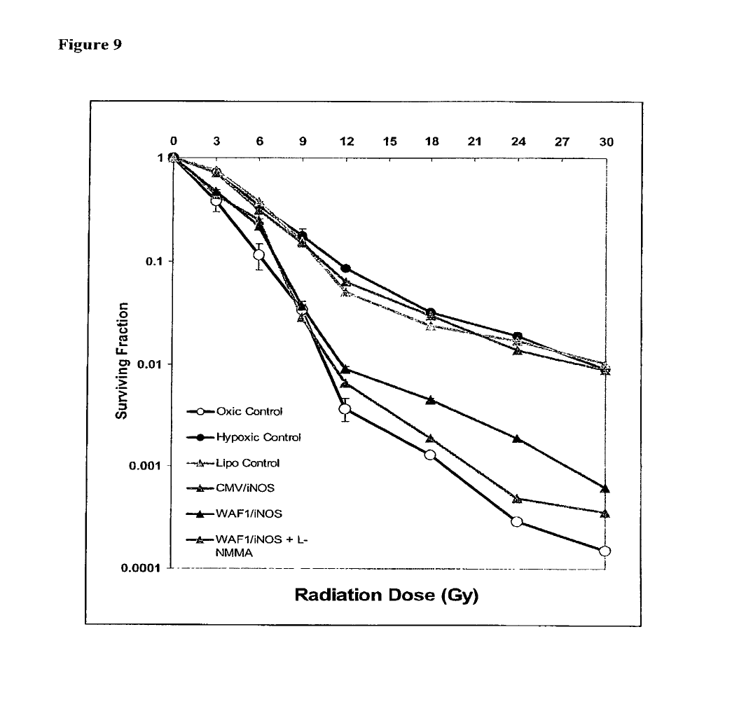

FIG. 9 shows the amount of iNOS expressed with WAF1 or CMV promoters and further the radiosensitization of RIF-1 tumour cells in vitro, and

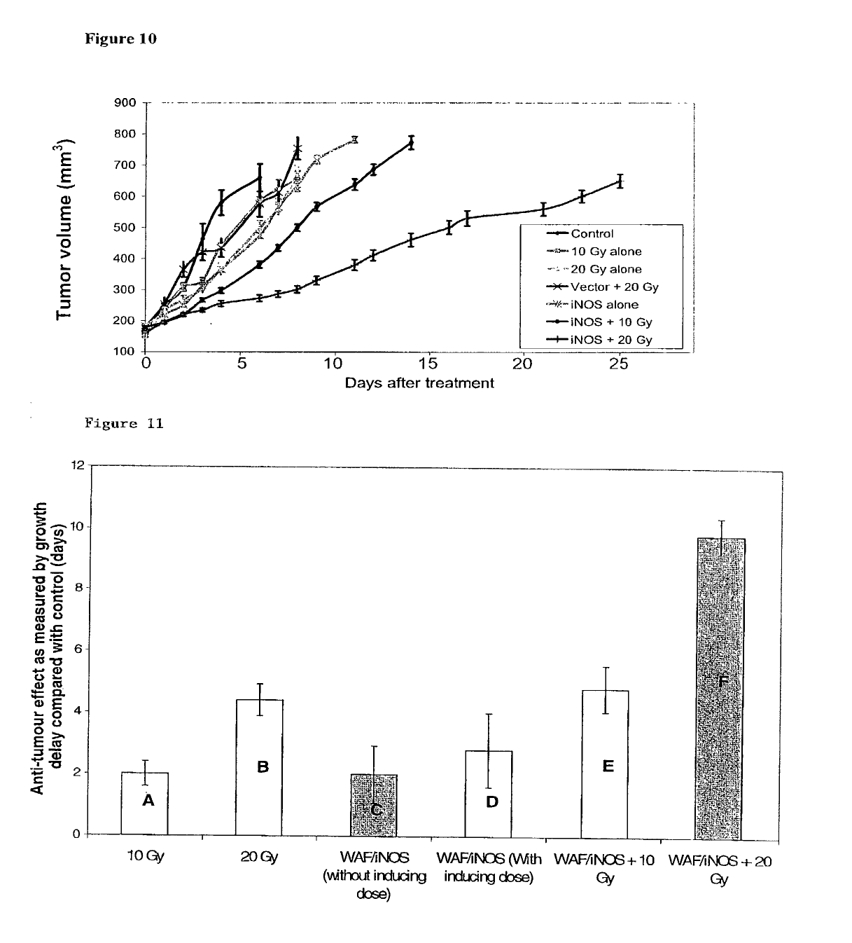

FIG. 10 shows the amount of iNOS with WAF1 or CMV promoters and further the radiosensitization of RIF-1 tumour cells in vivo.

FIG. 11 shows the results of Example 6.

Although the present embodiment discusses only the regulation of the expression of the enzyme inducible Nitric Oxide Synthase, other genes could be introduced into the vector such that they are regulated by the promoter of the X-ray inducible gene WAF1, and their expression modified by the provision of suitable radiation.

### EXAMPLE 1

### In-vitro Transfections with WAF1/pEGFP

Human micro-vascular endothelial cells (HMEC-1 cells) transfected with the WAF1/pEGFP reporter vector were irradiated with various doses of X-rays (0, 2, 4 and 6 Gy). At certain time points after irradiation (0, 30 min, 1, 2, 4, 8 and 24 hours), samples were extracted for western blot analysis. A dose of 2 Gy failed to induce WAF1 mediated GFP gene activation, however in cells exposed to 4 Gy of ionising radiation GFP levels rose rapidly.

FIG. 2 shows induction appearing after 1 hour and becoming statistically significant compared with background levels after 2 hours (P<0.05 (Annova)).

24 hours after irradiation, levels of GFP were 9.6 times higher than in unirradiated cells. Cells exposed to 6 Gy also showed a rise in GFP levels along the time course, with levels of GFP after 24 hours being 6.6 times higher than in cells which had not been irradiated

### EXAMPLE 2

### Ex-vivo Transfection of Rat Tail Artery with WAF1/pEGFP

The feasibility of transfecting rat tail artery in an ex-vivo perfusion apparatus was tested using the WAF1/pEGFP reporter vector. The artery was transfected for 2 hours, irradiated and at various time points (0, 1, 4 and 8 hours) after treatment a 1 cm³ section was removed for analysis of GFP levels using Western Blotting. Analysis could only be carried out up to a maximum of 8 hours after irradiation due to a finite life span of the artery ex-vivo.

FIG. 4 shows the induction of GFP expression in the rat tail artery segments over time.

Induction of GFP protein was observed after both 4 and 6 Gy.

By 8 hours post-irradiation GFP levels increased 4.5 and 8 fold respectively. This experiment established the principle that this perfusion apparatus could be used for future transfections using the WAF1/iNOS construct.

### EXAMPLE 3

### Ex-vivo Transfection of Rat Tail Artery with WAF1/iNOS construct

A section of rat tail artery was cannulated and placed on a perfusion apparatus. Its responsiveness was tested by perfusion with phenylephrine (PE). The artery was then transfected with the WAF1/iNOS vector, exposed to 4 Gy X-rays and its responsiveness to PE tested again at various time points.

FIG. 5 shows a typical computer trace of changes in pressure associated with testing the responsiveness of the artery at various times during the experiment.

At each of the time points (A, B, C, D & E) the artery was perfused with 2×10⁻⁵M PE in Krebs. During the initial test at time point A the artery had a normal contractile response to PE. The artery was then transfected, subjected to 4 Gy X-rays and tested again, with a normal response being observed (B). Time point C represents a responsiveness test 30 minutes after irradiation. The contractile response of the artery was reduced dramatically. At time point D, 60 minutes after exposure to X-rays, the artery was fully relaxed and exhibited almost no response to PE. The artery was then perfused for 1 hour with the iNOS inhibitor nitro-L-arginine and tested again with PE (E). This resulted in an increased, though not fully restored level of constriction.

As shown in FIG. 6, on average a significant decrease in responsiveness to PE was observed after 30 minutes, and by 60 minutes post-irradiation constriction of the artery was only 35% that of the control. Perfusion for one hour in the NOS inhibitor nitro-L-arginine resulted in constriction levels rising to 75% of control levels.

As described in FIG. 5 western blot analysis of homogenised artery samples taken at various time points (0, 1, 4 & 8 hours) after treatment with X-rays showed increased levels of iNOS protein at all doses. By 8 hours post-irradiation with 2 Gy, iNOS protein levels were 3.7 times above control levels, while after exposure to 4 and 6 Gy protein levels increased by 5.3 and 3.9 fold respectively. Although the unirradiated artery sample had a slightly higher level of iNOS protein than the control, which was neither transfected nor irradiated, this was not found to be statistically significant (P>0.05 (Annova)).

The above study found that artery tissue transfected with the vector construct, but not irradiated, showed no statistically significant increase in the level of iNOS (compared to non-transfected arteries). This suggests that the WAF1 promoter is minimally ‘leaky’ within the system.

Following an X-ray dose of 4 Gy to the in vitro samples, the WAF1 promoter induced similar levels of GFP and iNOS by a factor of 4.5 and 5.3 respectively.

Although the quantity of NO being released into the artery could not be measured in the above studies it was estimated that 5×10⁻⁷ mol/l of the nitric oxide donor sodium nitroprusside would be required to achieve a similar relaxation of the arteries as observed.

The use of liposomes to transfect the vector into cells has the intrinsic difficulty that he percentage of cells successfully transfected is low.

In an in vitro situation, using the GFP reporter vector attached to a constitutive promoter and visually estimating the number of cells transfected, it was found that a 23-27% transfection efficiency was achieved. However it would be expected that the percentage of cells transfected in the ex-vivo situation would be much lower. It has been reported in the literature that a 1% transfection efficiency of human iNOS into vascular cells can be achieved using a retroviral vector. Whilst for other gene therapy applications this low level of transfection efficiency would prove disastrous, for gene therapy involving the iNOS gene this does not represent a problem, as although the number of cells secreting the transgene product may be low, the vasoactive agent, NO is highly diffusable in solution and creates a ‘field effect’ around secreting cells. Therefore, even if iNOS is expressed in only a few cells, a wide field of cells could be exposed to NO.

Significantly enhanced levels (3.7 fold) of iNOS in arterial tissue following a dose of 2 Gy. Fractions of 2 Gy may be sufficient for treatment because NO generated from the radiation dose will further induce the WAF1 promoter, resulting in increased levels of iNOS being expressed, which will persist and accumulate during a course of therapy.

An alternative therapeutic regime would be to use an initial priming dose of 4 Gy, followed by subsequent 2 Gy fractions.

As an additional benefit of this approach, the increased levels of NO may act in a cytotoxic fashion.

Adopting this approach, it may be advantageous to incorporate another layer of specificity into the vector. In targeting the tumour only, the hypoxic environment within the tumour, could be exploited by choosing an enhancer element specific for hypoxia.

It has been shown that the WAF1 promoter is up-regulated in hypoxic conditions. Since only the vasculature is being targeted an obvious choice for an additional enhancer element would be the promoter region of the Vascular Endothelial Growth Factor, (VEGF) receptor (flt-1) which is expressed in proliferating endothelial cells.

However any suitable enhancer element regulating the expression of genes particular to proliferating endothelial cells could be utilised.

Although expression of Nitric Oxide can be expressed without the requirement of a cofactor to be present in the sample into which the vector, comprising the promoter for the X-ray inducible gene WAF1 and the sequence of the inducible gene to be regulated by WAF1, is introduced, it has been found that the addition of exogenous tetrahydrobioperin (BH₄) acts synergistically to cause an increase in the production of Nitric Oxide.

### EXAMPLE 4

Animal studies to investigate the use of the present invention to express NO at high concentrations within tumour cells have also been performed.

In an in vitro study RIF-1 tumour cells were transfected in vitro with either a CMV/iNOS construct or a WAF1/iNOS construct to achieve constitutive (continuous) or radiation-inducible (transient) expression respectively of iNOS protein at high levels within the cells. The medium was also supplemented with the co-factor tetrahydrobioperin (BH₄ 10⁻⁵M). Cells were first irradiated in air with a priming dose of 4 Gy (90 kV X-rays) which had previously been shown to give maximal induction of iNOS with the WAF1 promoter. This was followed 24 hours later by a range of radiation doses (3-30 Gy) under nitrogen or air. Clonogenic cell survival was calculated 12 days later.

FIG. 9 depicts the cell survival curve for irradiation under nitrogen and air.

This figure shows that CMV/iNOS transfected cells irradiated under nitrogen, were not significantly different from that of cells irradiated in air; the SER being 2.1. WAF1/iNOS transfection was slightly less effective, with a SER of 1.9.

The results of this in vitro study indicates that NO production by activation of the iNOS gene can greatly reduce the hypoxia-induced radioresistance in tumour cells.

### EXAMPLE 5

In an in vivo study Rif-1 tumour cells (2×10⁵) were injected intradermally on the rear dorsum of C3H mice and allowed to grow to form tumours of 7 mm mean diameter.

25 μg of the CMV/iNOS construct in a liposome-based vector was injected into the centre of the tumour mass in one group of animals. These mice were also injected intra-peritoneal. with 20 μl of 10⁻³ M BH₄. The tumours were then irradiated with 20 Gy (250 kv x-rays), together with a group of tumours that had not been injected with the construct. The tumours were measured daily then 3× weekly and growth curves plotted.

FIG. 10 shows the plotted growth curve.

The time for the tumours to reach 4× the volume at the time of the treatment was measured for each group: no treatment, 20 Gy alone and 20 Gy+iNOS gene therapy. Irradiation with 20 Gy alone gave a growth delay of 4±1.2 days compared with untreated tumours, whereas 20 Gy+iNOS gene therapy gave a delay of 12.3±3.5 days.

The results of this in vivo study indicates that NO production by activation of the iNOS gene sensitizes tumours to radiation in vivo.

These studies therefore describe the regulation of vascular tone through modulation of induction of an inducible gene under the control of the X-ray inducible promoter of the X-ray inducible gene WAF1. In particular the above data shows that a gene such as, but not limited to, the iNOS gene can be transfected into cells, tissues or organs under the control of the WAF1 promoter such that treatment of the transfected cells, tissues or organs with ionising radiation elicits a physiologically significant alteration in the concentration of the inducible gene.

Significantly enhanced levels (3.7 fold) of iNOS can be achieved in arterial tissue following a radiation dose of 2 Gy. Alternatively it is advantageous in stimulating iNOS to administer an initial priming dose of 4 Gy followed by subsequent 2 Gy fractions.

Increased Nitric Oxide levels due to stimulation of X-ray inducible promoters regulating the expression of transfected vectors encoding iNOS appear to result in increased blood flow and increased oxygenation within the tumour allowing a greater level of cell kill within the tumour by subsequent therapeutic doses of radiation while causing minimum damage to the surrounding tissue.

Alternatively chemotherapeutic agents could be delivered to the tumour through dilated blood vessels.

Further the increased levels of Nitric Oxide may act in a cytotoxic fashion.

### EXAMPLE 6

The effects of gene therapy using iNOS driven by the WAF1 promoter on the growth of RIF1 tumours were investigated. Tumours were directly injected with 25 μg of the transgene on day 1 and irradiated with a dose of 4 Gy X-rays 16 hours later (with the exception of group C) to induce the WAF1 promoter. Tumours were then irradiated with a therapeutic dose of 10 Gy (groups A and E), 20 Gy (groups B and F) or were sham irradiated (C). WAF1/iNOS transfection gave an enhancement ratio of >2.0 compared with radiation alone. Perhaps surprisingly WAF1/iNOS without the 4 Gy priming dose of radiation was as effective as WAF1/iNOS with the 4 Gy priming dose This suggests that WAF1 is being efficiently induced by the tumour environment (probably hypoxia) and emphasizes the value of the dual (radiation and hypoxia) inducibility of this promoter. By contrast previous data showed no leakiness of this promoter in endothelial cells.

The data shown in FIG. 11 supports the concept of the using radiation-inducible/hypoxia-inducible WAF1 promoter to kill cancer cells directly in vivo and to sensitize those that survive to the cell killing effects of radiotherapy.

Various modifications to the present invention can be made without departing from the scope of the invention, for example alternative genes could introduced into the vector such that they are regulated by promoter of the X-ray inducible gene WAF1, for example an appropriate antisense gene toward the gene encoding iNOS could be introduced into the vector. When the vector containing tumour cells are exposed to suitable radiation this would lead to a decrease in the levels of iNOS and thus in the levels of Nitric Oxide, such that there is a constriction in the tumour vessels and subsequent decreases in the oxygenation levels. This artificially hypoxic tumour environment could then be targeted with any novel bioreductive drugs such as AQ4N.

1. A method to regulate the expression of a sequence encoding a gene using ionising radiation, wherein said method comprises the steps of:
a. constructing a vector including the promoter of the X-ray inducible gene WAF1 and a sequence encoding an inducible gene the expression of which is regulated by the promoter of the X-ray inducible gene WAF1,
b. delivering said vector to the target cells, tissue or organ,
c. modulating the level of expression of the inducible gene(s) by providing ionising radiation suitable to cause the promoter of WAF1 to effect the expression of the sequence encoding the inducible gene.

2. A method as claimed in claim 1 further comprising the step wherein construct is delivered into cells by transfection.

3. A method as claimed in claim 1 wherein the expression of the sequence encoding the inducible gene is regulated by the promoter of the X-ray inducible gene WAF1 which encodes the inducible isoform of the Nitric Oxide Synthase enzyme.

4. A method as claimed in claim 1 wherein the expression of the sequence encoding the inducible gene is regulated by the promoter of the X-ray inducible gene WAF1 which encodes the antisense gene to the gene which encodes the inducible isoform of the Nitric Oxide Synthase enzyme.

5. A method as claimed in claim 1 wherein the level of expression of the sequence which encodes the inducible gene the expression of which is regulated by the promoter of the X-ray inducible gene WAF1 is modulated by providing X-rays at a clinically relevant dose.

6. A method as claimed in claim 1 wherein the level of expression of the sequence encoding the inducible gene is modulated by providing X-rays between the range 2 Gy-30 Gy.

7. A method as claimed in claim 1 wherein the level of expression of the sequence encoding the inducible gene is modulated by providing X-rays between the range 2 Gy-8 Gy.

8. A method as claimed in claim 1 wherein the method includes the step of administration of a cofactor.

9. A method as claimed in claim 8 wherein the cofactor is tetrahydrobiopterin or an equivalent thereof.

10. A vector comprising the promoter region for the X-ray inducible gene WAF1 and a sequence which encodes the inducible isoform of Nitric Oxide Synthase enzyme the expression of which is regulated by the promoter of the X-ray inducible gene WAF1.

11. A vector as claimed in claim 10 wherein the sequence which encodes for the inducible isoform of Nitric Oxide Synthase enzyme is an artificial sequence homologous to that of the naturally occurring gene.

12. A method of treatment of a tumour said method comprising the steps of,
a. introducing a vector including the promoter for the X-ray inducible gene WAF1, and a sequence encoding an inducible gene the expression of which is regulates by the promoter of the X-ray inducible gene WAF1 into the tumour,
b. irradiating the tumour such that the promoter for the X-ray inducible gene WAF1 induces sufficient levels of gene expression of the sequence for the inducible gene to provide physiological effect

13. A kit for use in the treatment of tumours wherein the kit comprises a vector including the promoter for the X-ray inducible gene WAF1 and a sequence encoding an inducible gene, the expression of which is regulated by the promoter of the X-ray inducible gene WAF1.

14. A kit as claimed in claim 13 wherein the kit comprises liposomes containing said vector such that the vector can be transfected into cells, tissues or organs.

15. A kit as claimed in claim 13 wherein the kit also comprises cofactors which aid the production of Nitric Oxide by inducible Nitric Oxide Synthase.

16. A kit as claimed in claim 15 wherein a cofactor included in the kit is the compound tetrahydrobiopterin, BH₄.
